(19) United States
(12) Patent Application Publication
(10) Pub. No.: US 2026/0183439 A1
(43) Pub. Date: Jul. 2, 2026

(54) Disinfection Scrub For Male And Female Luer Connectors

(71) Applicant: Becton, Dickinson and Company, Franklin Lakes, NJ (US)

(72) Inventors: Oya Okman, New York, NY (US); Richard Timmers, Westwood, NJ (US); Gheorghe Cojocariu, Bridgewater, NJ (US)

(73) Assignee: Becton, Dickinson and Company, Franklin Lakes, NJ (US)

(21) Appl. No.: 19/547,091

(22) Filed: Feb. 23, 2026

Related U.S. Application Data
(62) Division of application No. 16/923,238, filed on Jul. 8, 2020.
(60) Provisional application No. 62/873,470, filed on Jul. 12, 2019.

(51) Int. Cl. A61L 2/18; A61L 103/15; B32B 5/18; C08L 75/04

(52) U.S. Cl. CPC A61L 2/18; B32B 5/18; A61L 2103/15; A61L 2202/121; A61L 2202/182; B32B 2535/00; C08L 75/04

(57) ABSTRACT

A disinfection device is disclosed having a container, a scrub element, a disinfectant or antimicrobial agent, and a removable seal. The scrub element may be disposed on a base scrub. The container configured to define a chamber to contain the base scrub, scrub element, and disinfectant or antimicrobial agent. The base scrub and/or scrub element(s) are adapted to compress and contact the distal tip and sidewall of a male Luer connector, female Luer connector or hemodialysis connector upon insertion of the connector into the chamber. The removable seal prevents the disinfectant or the antimicrobial agent from exiting the chamber. Also described are methods of disinfecting a medical connector, and an assembly comprising a device for disinfecting a medical connector.

### CROSS-REFERENCE TO RELATED APPLICATIONS

This application is a divisional of U.S. patent application Ser. No. 16/923,238, filed on Jul. 8, 2020, which claims priority under 35 U.S.C. § 119(e) to U.S. Provisional Application No. 62/873,470, filed Jul. 12, 2019, the disclosures of which are incorporated herein by reference in their entirety.

### TECHNICAL FIELD

The present disclosure generally relates to a disinfection device for disinfecting injection hubs and access ports with, e.g., male and female Luer fitting. The disinfection device is capable of accommodating multiple types of connectors. The disinfection device may be integrated into caps, port disinfection devices, and integrated disinfection units of syringes.

### BACKGROUND

Vascular access devices (VADs) are commonly used therapeutic devices and include intravenous (IV) catheters, injection hubs and access ports with, e.g., male and female Luer fitting. Bacteria and other microorganisms may gain entry into a patient's vascular system from access hubs and ports/valves upon connection to the VAD to deliver the fluid or pharmaceutical. Each access hub (or port/valve or connection) is associated with some risk of transmitting a catheter related bloodstream infection (CRBSI), which can be costly and potentially lethal.

In order to decrease CRBSI cases and to ensure VADs are used and maintained correctly, standards of practice have been developed, which include disinfecting and cleaning procedures.

Disinfection devices have been added to the Society for Healthcare Epidemiology of America (SHEA) guidelines. Disinfection devices may also be incorporated into the Infusion Nurses Standards (INS) guidelines.

In developed markets, when utilizing an IV catheter, a needleless connector will typically be used to close off the system and then subsequently accessed to administer medication or other necessary fluids via the catheter to the patient. Infusion Nurses Standards (INS) Standards of Practice recommend the use of a needleless connector and state that it should be “consistently and thoroughly disinfected using alcohol, tincture of iodine or chlorhexidine gluconate/alcohol combination prior to each access.” The disinfection of the needleless connector is ultimately intended to aid in the reduction of bacteria that could be living on the surface and possibly lead to a variety of catheter related complications including CRBSI. Nurses will typically utilize a 70% isopropyl alcohol (IPA) swab to complete this disinfection task by doing what is known as “scrubbing the hub.” However, compliance to this practice varies from user to user, thus resulting in inconsistent disinfection practices and results depending on the user. In addition to a lack of compliance to “scrubbing the hub”, it has also been noted through clinician interviews that there is often a variation in scrub time, dry time and the number of times the needleless connector is scrubbed.

However, disinfection devices currently available fail to contact the entire face and sides of an injection port at all times during the swabbing.

Therefore, there is a need for a disinfection device that simultaneously contacts the entire face and sides of an injection port at all times during the swabbing. There is a need for a disinfection device that wherein the disinfection practice is regulated by product design rather than the user's ability. There is a need for a disinfection device that relatively inexpensive to manufacture.

### SUMMARY

One aspect of the present disclosure pertains to a disinfection device for connection to a medical connector. According to an exemplary embodiment of the present disclosure, a device generally comprises a container, a scrub element, a disinfectant or an antimicrobial agent, and a removable seal. In one or more embodiments, the disinfection device also comprises an scrub base wherein the scrub element is disposed onto the scrub base. In one or more embodiments, the scrub base is made from an absorbent material. The container comprises an integral body, a closed end, an annular wall having a length extending from the closed end to an open end that defines a chamber containing a scrub element and disinfectant or antimicrobial agent. The open end defines an engagement surface.

The annular wall of the container comprises an exterior wall surface and an interior wall surface. The interior wall surface defines an opening adjacent the open end.

The scrub element and the disinfectant or the antimicrobial agent contacts the male Luer connector, the female Luer connector and the hemodialysis connector after insertion of the connector into the container.

The removable seal can be disposed on the end face of the container to prevent the disinfectant or the antimicrobial agent from exiting the chamber prior to use.

The container can be made from any of a number of types of plastic materials such as polycarbonate, polypropylene, polyethylene, glycol-modified polyethylene terephthalate, acrylonitrile butadiene styrene or any other moldable plastic material used in medical devices. In one or more embodiments, the container comprises a polypropylene or polyethylene material. In one or more embodiments, the exterior container surface includes a plurality of grip members.

In one or more embodiments, the scrub element has slits. In one or more embodiments, the scrub element includes an annular wall, an exterior wall surface, an interior wall surface, an inner cavity, a bottom surface, a top surface, and a single slit that extends vertically from the bottom surface to the top surface and radially outward from the interior wall surface to the exterior wall surface. In an alternate embodiment, the scrub element includes an annular wall, an exterior wall surface, an interior wall surface, an inner cavity, a bottom surface, a top surface, and a single semi-circular slit. In a specific embodiment, the single semi-circular slit partially revolves around an x-axis about a distance y from an origin of the top surface of the scrub element. In another alternate embodiment, the scrub element includes an annular wall, an exterior wall surface, an interior wall surface, an inner cavity, a bottom surface, a top surface, and two or more semi-circular slits that are asymmetrically oriented. In one or more embodiments, the two or more semi-circular slits are oriented as mirror images. In one or more embodiments, the two or more semi-circular slits are not superimposed.

In one or more embodiments, the scrub element is made of a nonwoven material, foam or a sponge. In a specific embodiment, the foam is a polyurethane foam.

In one or more embodiments, the disinfectant or antimicrobial agent is selected from the group consisting essentially of isopropyl alcohol, ethanol, 2-propanol, butanol, methylparaben, ethylparaben, propylparaben, propyl gallate, butylated hydroxyanisole (BHA), butylated hydroxytoluene, t-butyl-hydroquinone, chloroxylenol, chlorohexidine, chlorhexidine diacetate, chlorohexidine gluconate, povidone iodine, alcohol, dichlorobenzyl alcohol, dehydroacetic acid, hexetidine, triclosan, hydrogen peroxide, colloidal silver, benzethonium chloride, benzalkonium chloride, octenidine, antibiotic, and mixtures thereof. In a specific embodiment, the disinfectant or antimicrobial agent comprises at least one of chlorhexidine gluconate and chlorhexidine diacetate. In one or more embodiments, the disinfectant or antimicrobial agent is a fluid or a gel.

Compression of the scrub element toward the closed end of the chamber upon connection to the female Luer connector or the male Luer connector allows the connector to contact the disinfectant or antimicrobial agent to disinfect the female Luer connector or the male Luer connector.

In one or more embodiments, the removable seal comprises an aluminum or multi-layer polymer film peel back top. In a specific embodiment, the removable seal is heat-sealed or induction sealed to the end face of the container.

A second aspect of the present disclosure pertains to a method of disinfecting a medical connector. The method comprises connecting the device of one or more embodiments to a medical connector, wherein connecting includes engaging the interior wall surface upon insertion into the chamber such that the medical connector contacts the scrub element and the disinfectant or antimicrobial agent.

In one or more embodiments, the medical connector is selected from a male Luer connector, a female Luer connector, and needleless connector.

### BRIEF DESCRIPTION OF THE DRAWINGS

FIG. 1 shows a perspective view of a first embodiment of a device of the present disclosure;

FIG. 2 shows a perspective view of an exemplary scrub element of the first embodiment of the present disclosure;

FIG. 3 shows a perspective view of an exemplary absorbent base of the first embodiment of the present disclosure;

FIG. 4 shows a perspective view of an exemplary connector that may be disinfected by one or more embodiments of the disinfection device of the present disclosure;

FIG. 5 shows a cross sectional view of the device in accordance with the first embodiment of FIG. 1;

FIG. 6 shows an alternative embodiment of the scrub element of the present disclosure; and,

FIG. 7 shows yet another alternative embodiment of the scrub element of the present disclosure.

### DETAILED DESCRIPTION

Before describing several exemplary embodiments of the disclosure, it is to be understood that the disclosure is not limited to the details of construction or process steps set forth in the following description. The disclosure is capable of other embodiments and of being practiced or being carried out in various ways.

Embodiments of the disclosure pertain to a disinfection device for application to and disinfection of a medical connector, male Luer connectors and female Luer connectors, in which the device comprises a side/thread scrub element, base scrub element, a disinfection cap, disinfectant or the antimicrobial agent and a removable seal. The scrub elements may or may not be absorbent. The disinfection device of the present disclosure provides a mechanical scrubbing element for connectors and contains an antimicrobial agent for disinfection. The device of the present disclosure allows the practitioner to streamline the disinfecting process.

With respect to terms used in this disclosure, the following definitions are provided.

As used herein, the use of “a,” “an,” and “the” includes the singular and plural.

As used herein, the term “catheter related bloodstream infection” or “CRBSI” refers to any infection resulting from the presence of a catheter or IV line.

As used herein, the term “Luer connector” refers to a connection collar that is the standard way of attaching syringes, catheters, hubbed needles, IV tubes, etc. to each other. The Luer connector consists of male and female interlocking tubes, slightly tapered to hold together better with even just a simple pressure/twist fit. Luer connectors can optionally include an additional outer rim of threading, allowing them to be more secure. The Luer connector male end can interlock and connect to the female end located on the vascular access device (VAD). A Luer connector comprises a distal end, a proximal end, an irregularly shaped outer wall, a profiled center passageway for fluid communication from the chamber of the barrel of a syringe to the hub of a VAD. A Luer connector also has a distal end channel that releasably attaches the Luer connector to the hub of a VAD, and a proximal end channel that releasably attaches the Luer connector to the barrel of a syringe.

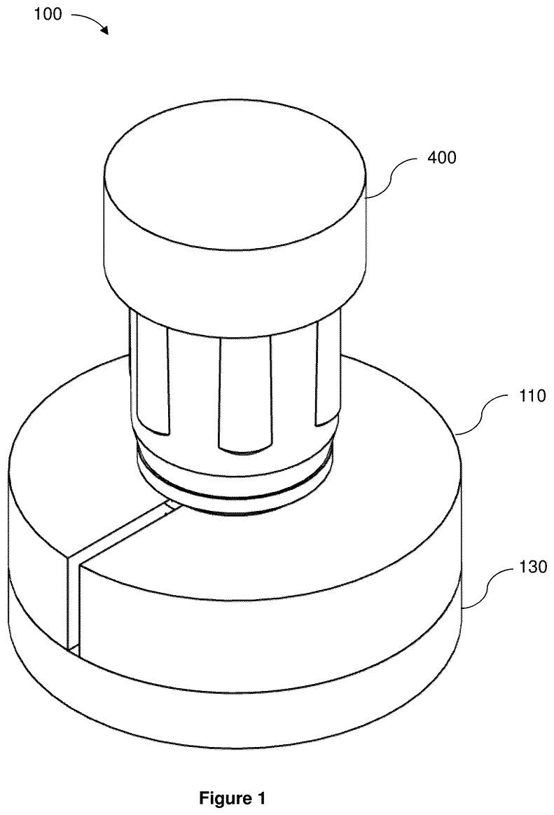

Each of FIGS. 1-7, wherein like reference characters indicate like parts, displays different configurations in which a scrub element 110 can be molded or extruded to form different shapes for various connectors.

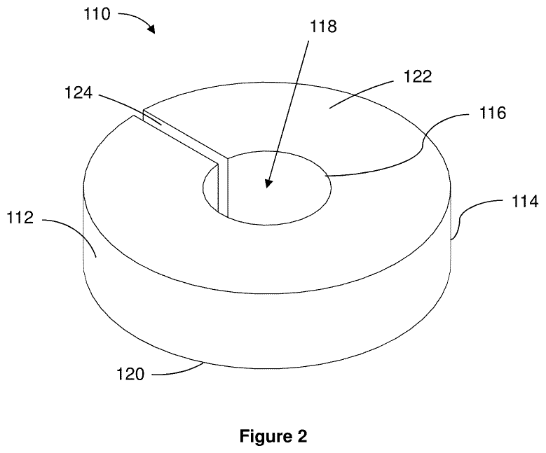

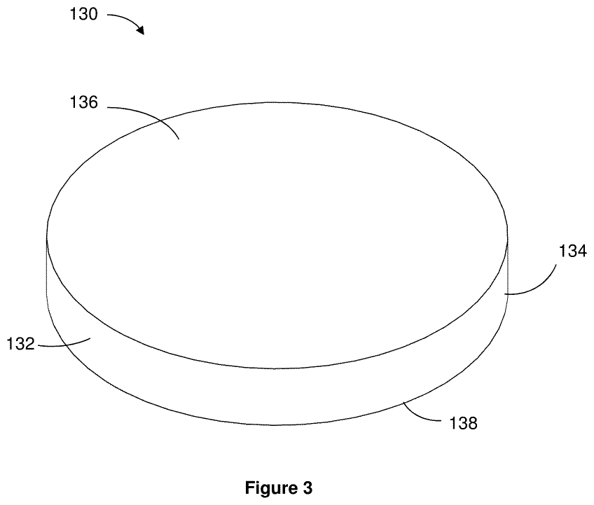

A disinfection device 100 according to a first embodiment is shown in FIG. 1, with the components shown separately in FIGS. 2 and 3. Referring to FIG. 1, a disinfection device 100 for a disinfectant scrub of various types of medical connectors and other medical device protrusions according to an exemplary embodiment of the present disclosure generally comprises side scrub element 110, a base scrub element 130, and a disinfectant or an antimicrobial agent. An exemplary embodiment of a medical connector 400 is shown in FIGS. 1, 4 and 5 for illustration purposes only.

Referring to FIG. 2, a first embodiment of the side scrub element 110 comprises of an annular wall 112, an exterior wall surface 114, an interior wall surface 116, an inner cavity 118, a bottom surface 120, a top surface 122, and a single slit 124. The interior wall surface 116 defines the inner cavity 118 that extends from the bottom surface 120 to the top surface 122. The scrub element 110 may be soaked with a disinfectant or an antimicrobial agent. In one or more embodiments the scrub element 110 is a nonwoven material, foam, or a sponge. In a specific embodiment, the foam is a polyurethane foam. In one or more embodiments, the scrub element 110 is molded or extruded or die cut from sheeting to form a cylindrical block shape. In particular, the scrub element 110 is injection molded.

The scrub element 110 also comprises of a slit 124 that extends vertically from the bottom surface 120 to the top surface 122 and radially outward from the interior wall surface 116 to the exterior wall surface 114. As shown in FIG. 2, in one or more embodiments, scrub element 110 is “C-shaped” due to the presence of slit 124. An added advantage of the scrub element 110 of the present disclosure is that during manufacturing, the slit 124 in the scrub element 110 can be cut using a single die and in a single stroke thus providing a more robust and cheaper alternative to cutting concentric circles in two or more steps, which requires precise alignment, and increases operation time. As additional advantage is that during use, the center portion of the scrub element 110 is depressed entirely during the cleaning without separating or detaching from the interior wall surface.

Referring to FIG. 3, in one or more embodiments, scrub element 110 is disposed onto a base scrub element 130. Base scrub element 130 comprises an integral body 132, an annular wall 134, a disinfecting surface 136, and a bottom 138. The base scrub 130 may be soaked with a disinfectant or an antimicrobial agent. In one or more embodiments the base scrub 130 is a nonwoven material, foam, or a sponge. In a specific embodiment, the foam is a polyethylene foam. The foam may be open celled, semi-opened or closed celled and may be molded or extruded or die cut from sheeting. In one or more embodiments, the base scrub 130 is molded or extruded or die cut from sheeting to form a cylindrical block shape. In particular, the base scrub 130 is die cut from sheeting.

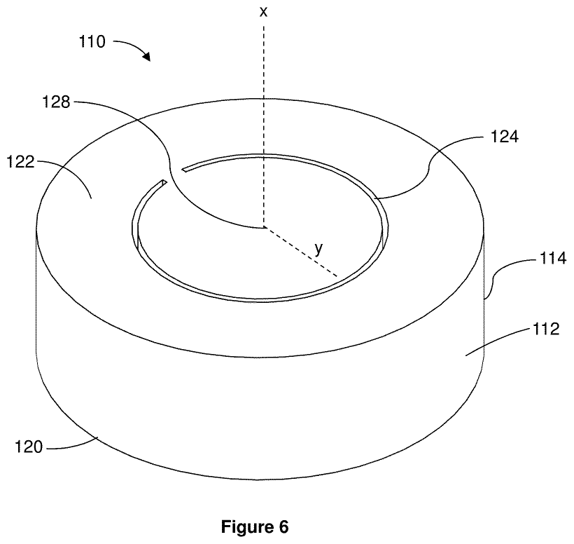

An alternate embodiment of the scrub element 110 is shown in FIG. 6. FIG. 6 shows the scrub element 110 having an annular wall 112, a bottom surface 120, and single semi-circular arc slit 124 disposed in the center of top surface 122. The annular wall 112 of the scrub element 110 comprises of an exterior wall surface 114. The top surface 122 of the scrub element 110 comprises of a single semi-circular arc slit 124 made within the body of the scrub element 110. The single semi-circular arc slit 124 revolves around x-axis about a distance y from the origin 128 of the top surface 122 of the scrub element 110; however, the single semi-circular arc slit 124 does not make a complete revolution about the x-axis. Thus, as shown in FIG. 6, the single semi-circular arc slit 124 of the scrub element 110 is in the shape of a “C”. The concentric slit 124 extends vertically from the bottom surface 120 to the top surface 122 of the scrub element 110.

The angle of revolution for the single semi-circular arc slit 124 about the x-axis may vary. The distance Y of the single semi-circular arc slit 124 from the origin 128 within the scrub element 110 can include any value less than the radius of the scrub element 110. As additional advantage is that during use, the center portion of the scrub element 110 is depressed entirely during the cleaning without separating or detaching from the interior wall surface. In one or more embodiments, scrub element 110 having a single semi-circular arc slit is disposed onto a base scrub 130. In one or more embodiments, base scrub 130 is made of an absorbent material.

In one or more embodiments, the scrub element 110 may be housed in a container so to prevent escape of the disinfectant or the antimicrobial agent soaked into the scrub element 110. In one or more embodiments, the container may be a cap, port disinfection device, or integrated disinfection unit of a syringe assembly.

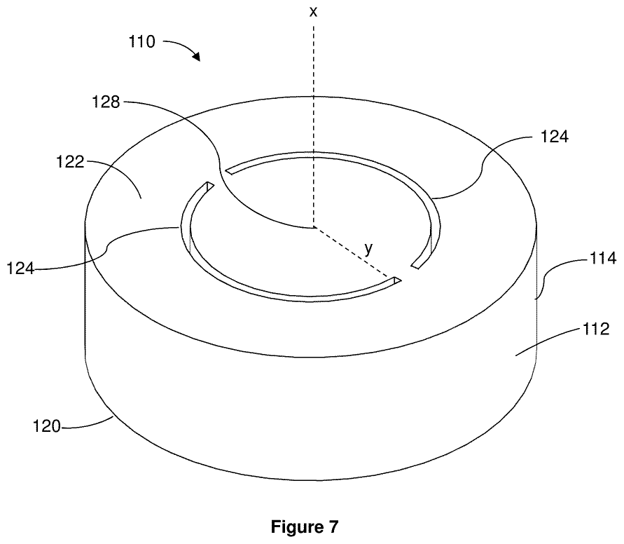

Yet another alternate embodiment of the scrub element 110 is shown in FIG. 7. FIG. 7 shows the scrub element 110 having two or more concentric or semi-circular slits 124. The scrub element 110 comprises of an annular wall 112, a bottom surface 120, and a top surface 122 having two or more concentric or semi-circular slits 124. The annular wall 112 of the scrub element 110 comprises of an exterior wall surface 114.

In one or more embodiments, the scrub element 110 may be housed in a container so to prevent escape of the disinfectant or the antimicrobial agent soaked into the scrub element 110. In one or more embodiments, the container may be a cap, port disinfection device, or integrated disinfection unit of a syringe assembly.

As shown in FIG. 7, the top surface 122 of the scrub element 110 comprises of two or more concentric or semi-circular slits 124 made within the body of the scrub element 110. In one or more embodiments, the two or more concentric or semi-circular slits 124 revolve around the x-axis about a distance y from the origin 128 of the top surface 122 of the scrub element 110. In one or more embodiments, the two or more concentric or semi-circular slits 124 do not make a complete revolution about x-axis. In a specific embodiment, as shown in FIG. 7, the two or more slits 124 are semi-circular. In one or more embodiments, as shown in FIG. 7, the concentric slits 124 are asymmetrically oriented in such a way that the concentric slits 124 are mirror images are not superimposed on one another. In one or more embodiments, the concentric slits 124 extend vertically from the bottom surface 120 to the top surface 124 of the scrub element 110. In one or more embodiments, the concentric slits 124 may extend partially in a vertical direction from the top surface 122 to the bottom surface 120 of the scrub element 110. In one or more embodiments, the concentric slits 124 may extend fully in a vertical direction from the top surface 122 to the bottom surface 120 of the scrub element 110.

The angle of revolution for the concentric slits 124 about the x-axis may vary. The distance Y the concentric slits 124 is made from the origin 128 within the scrub element 110 can include any value less than the radius of the scrub element 110.

During use, the center portion of the scrub element 110 is depressed entirely during the disinfection. As additional advantage is that during use, the center portion of the scrub element 110 is depressed entirely during the cleaning without separating or detaching from the interior wall surface. In one or more embodiments, scrub element 110 having a single semi-circular arc slit is disposed onto a base scrub 130. In one or more embodiments, base scrub 130 is made of an absorbent material. In one or more alternate embodiment, the base scrub is made of a closed cell foam.

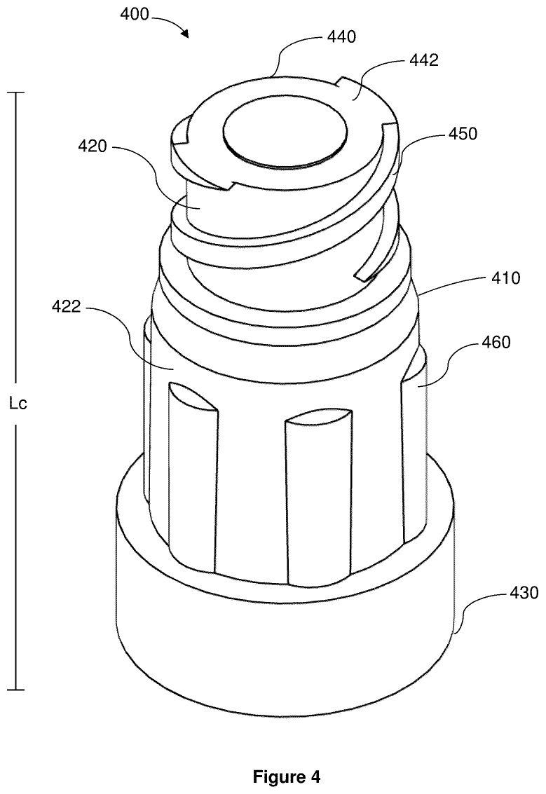

FIG. 4 shows an exemplary medical connector 400 comprises of an integral body 410, an annular sidewall 420, a proximal end 430, and a distal end 440 having a distal tip 442. The annular sidewall 420 may include threads 450. The medical connector 400 has a length L_{C} extending from the proximal end 430 to the distal end 440. The annular sidewall 420 of the medical connector 400 comprises of an exterior wall surface 422. The exterior wall surface 422 of annular sidewall 420 may comprise of external threads 450 adjacent to the distal end 440. The external threads 450 are adapted and sized to engage a female Luer connector. The exterior wall surface of integral body 410 may contain a plurality of protrusions 460 used for grip when twisting or turning the medical connector 400.

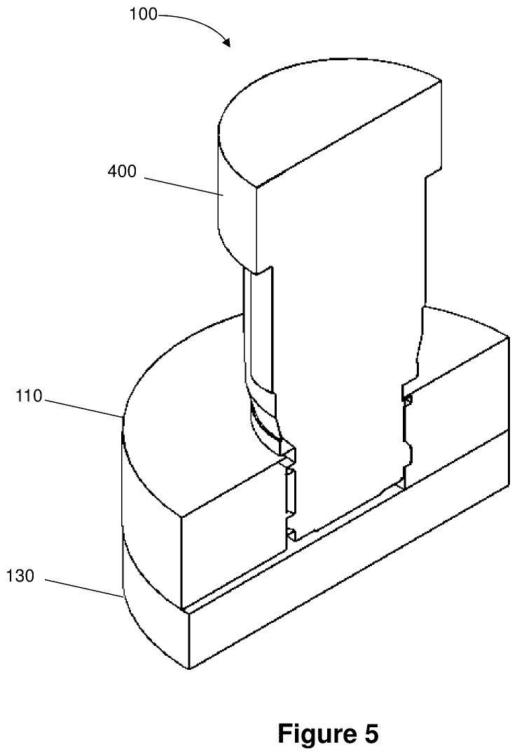

As shown in FIG. 1 and FIG. 5, the scrub element 110 can be sized to frictionally engage the distal end 440 of medical connector 400. In one or more embodiments, the distal end 440 of medical connector 400 frictionally engages the scrub element 110 via a press-fit connection upon insertion into the inner cavity 118 of the scrub element 110. Once inserted, the interior wall surface 116 of the scrub element 110 makes frictional contact with the annular sidewall 420 and threads 450 of the medical connector 400. Additionally, the disinfecting surface 136 of the base scrub 130 makes frictional contact with the distal end 440 and threads 450 of the medical connector 400. Thus, scrub element 110 can exert mechanical force on the distal face and annular sidewall and threads of a connector throughout scrubbing. Medical connector 400 may include male Luer connectors, female Luer connectors, and hemodialysis connectors.

Use of the disinfection device 100 requires only one single mounting movement by a user. Upon insertion of the medical connector 400 into disinfection device 100, the user may twist or compress the medical connector 400 to promote disinfection of the medical connector 400. Use of the disinfection device 100 does not activate the fluid path of a female Luer connector having a septum or a hemodialysis connector having a sheath.

The scrub element 110 and the disinfectant or the antimicrobial agent contacts the external threads 450 of the medical connector 400 after insertion of the medical connector 400 into the inner cavity 118 of the scrub element 110.

In one or more embodiments, the scrub element 110 and the base scrub 130 may be housed in a container so to prevent escape of the disinfectant or the antimicrobial agent soaked into the scrub element 110 or the base scrub 130. In one or more embodiments, the container may be a cap, port disinfection device, or integrated disinfection unit of a syringe assembly.

In one or more embodiments, the container that houses the scrub element 110 and base scrub 130 may have an end surface where a removable seal may be disposed to prevent the disinfectant or the antimicrobial agent from exiting the chamber of a container. With the scrub element 110 and base scrub 130 properly inserted into the chamber of the container, the removable seal may be secured to the end surface of the container to seal the container. The removable seal minimizes entry of potential particulate hazard and also provides a substantially impermeable enclosure for the scrub element 110, provides a leak prevention and protection enclosure, protects the contents of scrub element 110 contained within the chamber, and/or maintains a sealed, sterilized environment. A removable seal provides a sufficient seal at a range of temperatures, pressures, and humidity levels.

The scrub element 110 and the disinfectant or the antimicrobial agent contacts the male Luer connector, the female Luer connector, and the hemodialysis connector after insertion of the connector into the open end of a container.

Medical connector 400 may include male Luer connectors, female Luer connectors, and hemodialysis connectors. In one or more embodiments, the female Luer connector may be selected from the group consisting essentially of needle-free connectors, catheter Luer connectors, stopcocks, and hemodialysis connectors. In one or more embodiments, the needleless connector is selected from a Q-Syte connector, MaxPlus, MaxPlus Clear, MaxZero, UltraSite, Caresite, InVision-Plus, Safeline, OneLink, V-Link, ClearLink, NeutraClear, Clave, MicroClave, MicroClave Clear, Neutron, NanoClave, Kendall, Nexus, InVision, Vadsite, Bionector, etc. In one or more embodiments, the male Luer connector may be an intravenous tubing end, a stopcock or male lock Luer.

The container can be made from any of a number of types of plastic materials such as polycarbonate, polypropylene, polyethylene, polyethylene terephthalate, polylactide, acrylonitrile butadiene styrene or any other moldable plastic material used in medical devices. In one or more embodiments, the container comprises a polypropylene or polyethylene material.

Referring to FIGS. 1 and 5, in one or more embodiments, upon initial insertion of a medical connector 400 into the disinfection device 100, the scrub element 110 is under radial compression by the distal end 440 of the medical connector 400. Upon further insertion of a medical connector 400 into the inner cavity of the scrub element, the interior wall surface 116 of the scrub element 110 envelopes the annular sidewall 420 and threads 450 disposed on the distal end 440 of medical connector 400. In one or more embodiments, upon further insertion of a medical connector 400 into the inner cavity of the scrub element, the distal end 442 contacts base scrub 130 and/or single semi-circular arc slit 124 from the origin 128 within the scrub element 110 for disinfection. In one or more embodiments, the scrub element 110 is a nonwoven material, foam, or a sponge. In a specific embodiment, the foam is a polyurethane foam. In one or more embodiments, the scrub element 110 includes one or more slits 124.

The disinfection device 100 can achieve disinfection when used on medical connectors, including needless connectors or Luer connectors, by integrating disinfectant or antimicrobial agent into scrub element 110 and base scrub 130 in the chamber of the container. The disinfectant or antimicrobial agent can be directly included in the chamber or disinfectant or antimicrobial agent can be absorbed into sponges or foam material that fills the chamber of container. The disinfection device 100 is designed to be compatible in interacting with various disinfectants. In one or more embodiments, the disinfectant or antimicrobial agent may include variations of alcohol or chlorhexidine. In one or more embodiments, the disinfectant or antimicrobial agent is selected from the group consisting essentially of isopropyl alcohol, ethanol, 2-propanol, butanol, methylparaben, ethylparaben, propylparaben, propyl gallate, butylated hydroxyanisole (BHA), butylated hydroxytoluene, t-butyl-hydroquinone, chloroxylenol, chlorohexidine, chlorhexidine diacetate, chlorohexidine gluconate, povidone iodine, alcohol, dichlorobenzyl alcohol, dehydroacetic acid, hexetidine, triclosan, hydrogen peroxide, colloidal silver, benzethonium chloride, benzalkonium chloride, octenidine, antibiotic, and mixtures thereof. In a specific embodiment, the disinfectant or antimicrobial agent comprises at least isopropyl alcohol. In one or more embodiments, the disinfectant or antimicrobial agent is a fluid or a gel.

In one or more embodiments, the removable seal may comprise an aluminum or multi-layer polymer film peel back top. In a specific embodiment, the removable seal is heat-sealed or induction sealed to the end face of the container or distal end of the disinfection device. In one or more embodiments, the removable seal comprises a moisture barrier.

The disinfection device 100 of the present disclosure minimizes ingress of microbial agents.

Other aspects of the present disclosure are directed to methods of disinfecting medical connectors and assemblies. In one or more embodiments, a method of disinfecting a medical connector comprises contacting the disinfection device 100 of one or more embodiments to a medical connector 400, wherein contacting includes frictionally engaging and mechanically scrubbing the distal end 442 and threads 450 of the medical connector 400 with the scrub element 110 and/or base scrub 130 upon insertion into a chamber of a container such that the medical connector contacts the scrub element and the disinfectant or antimicrobial agent.

Embodiments of the disinfection devices of the present disclosure simultaneously contact the entire face and sides of a medical connector at all times during the swabbing. The force exerted during mechanical scrubbing by embodiments of the disinfection devices of the present disclosure are regulated by the design of the disinfection device 100, rather than user's ability.

An additional benefit is that embodiments of the scrub element 110 of the disinfection devices of the present disclosure is that the one or more concentric arc slits 124 in the scrub element 110 can be cut using a single die and in a single stroke. Thus, the embodiments of the scrub element 110 of the disinfection devices of the present disclosure provide a more robust and cheaper alternative to cutting concentric circles in two or more steps, which requires precise alignment, and increases operation time. Therefore, embodiments of the scrub element 110 of the disinfection devices of the present disclosure reduce die costs and eliminating possible diameter alignment issues thus resulting in lower cost of manufacture.

Reference throughout this specification to “one embodiment,” “certain embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein has provided a description with reference to particular embodiments, it is to be understood that these embodiments are merely illustrative of the principles and applications of the present disclosure. It will be apparent to those skilled in the art that various modifications and variations can be made to the method and apparatus of the present disclosure without departing from the spirit and scope of the disclosure. Thus, it is intended that the present disclosure include modifications and variations that are within the scope of the appended claims and their equivalents.

What is claimed is:

1. A disinfection device comprising:
a container comprising an integral body, an open end, a closed end, an annular wall defining a chamber extending from the closed end to an open end;
a scrub element having an annular wall, an exterior wall surface, an interior wall surface, an inner cavity, a bottom surface, a top surface, and a single semi-circular slit;
a disinfectant or antimicrobial agent; and
a removable seal on the open end of the container.

2. The disinfection device of claim 1, wherein the single semi-circular slit partially revolves around an x-axis about a distance y from an origin of the top surface of the scrub element.

3. The disinfection device of claim 1, wherein the scrub element is a foam.

4. The disinfection device of claim 3, wherein the foam is a polyurethane foam.

5. The disinfection device of claim 1, wherein the scrub element is a sponge.

6. The disinfection device of claim 5, wherein a compression of the scrub element toward the closed end of the chamber occurs upon contact to a female Luer connector or a male Luer connector.

7. The disinfection device of claim 6, wherein compression of the scrub element disinfects the female Luer connector or the male Luer connector.

8. The disinfection device of claim 1, wherein the disinfectant or antimicrobial agent is selected from a group consisting essentially of isopropyl alcohol, ethanol, 2-propanol, butanol, methylparaben, ethylparaben, propylparaben, propyl gallate, butylated hydroxyanisole (BHA), butylated hydroxytoluene, t-butyl-hydroquinone, chloroxylenol, chlorohexidine, chlorhexidine diacetate, chlorohexidine gluconate, povidone iodine, alcohol, dichlorobenzyl alcohol, dehydroacetic acid, hexetidine, triclosan, hydrogen peroxide, colloidal silver, benzethonium chloride, benzalkonium chloride, octenidine, antibiotic, and mixtures thereof.

9. The disinfection device of claim 1, wherein the disinfectant or antimicrobial agent is a fluid or a gel.

10. The disinfection device of claim 1, wherein the removable seal comprises an aluminum or multi-layer polymer film peel back top.

11. The disinfection device of claim 1, wherein the removable seal is heat-sealed or induction sealed to an engagement surface on the open end of the container.

12. A method of disinfecting a medical connector, the method comprising: connecting the disinfection device of claim 1 to a medical connector, wherein connecting includes engaging the medical connector to the interior wall surface of the scrub element upon insertion into the chamber such that the medical connector contacts the scrub element and the disinfectant or antimicrobial agent.

13. A disinfection device comprising:
a container comprising an integral body, an open end, a closed end, an annular wall defining a chamber extending from the closed end to an open end;
a scrub element having an annular wall, an exterior wall surface, an interior wall surface, an inner cavity, a bottom surface, a top surface, and two or more semi-circular slits that are asymmetrically oriented;
a disinfectant or antimicrobial agent; and
a removable seal on the open end of the container.

14. The disinfection device of claim 13, wherein the container comprises a polypropylene or polyethylene material.

15. The disinfection device of claim 13, wherein the two or more semi-circular slits are oriented as mirror images.

16. The disinfection device of claim 15, wherein the two or more semi-circular slits are not superimposed.

17. The disinfection device of claim 13, wherein the scrub element is a foam.

18. The disinfection device of claim 17, wherein the foam is a polyurethane foam.

19. The disinfection device of claim 13, wherein the scrub element is a sponge.

20. The disinfection device of claim 13, wherein a compression of the scrub element toward the closed end of the chamber occurs upon connection to a female Luer connector or a male Luer connector.

21. The disinfection device of claim 20, wherein compression of the scrub element disinfects the female Luer connector or the male Luer connector.

22. The disinfection device of claim 13, wherein the disinfectant or antimicrobial agent is selected from a group consisting essentially of isopropyl alcohol, ethanol, 2-propanol, butanol, methylparaben, ethylparaben, propylparaben, propyl gallate, butylated hydroxyanisole (BHA), butylated hydroxytoluene, t-butyl-hydroquinone, chloroxylenol, chlorohexidine, chlorhexidine diacetate, chlorohexidine gluconate, povidone iodine, alcohol, dichlorobenzyl alcohol, dehydroacetic acid, hexetidine, triclosan, hydrogen peroxide, colloidal silver, benzethonium chloride, benzalkonium chloride, octenidine, antibiotic, and mixtures thereof.

23. The disinfection device of claim 13, wherein the disinfectant or antimicrobial agent is a fluid or a gel.

24. The disinfection device of claim 13, wherein the removable seal comprises an aluminum or multi-layer polymer film peel back top.

25. The disinfection device of claim 13, wherein the removable seal is heat-sealed or induction sealed to an engagement surface on an open end of the container.

26. A method of disinfecting a medical connector, the method comprising: contacting the disinfection device of claim 13 to a medical connector, wherein contacting includes engaging the medical connector to the interior wall surface of the scrub element upon insertion into the chamber such that the medical connector frictionally engages the scrub element and the disinfectant or antimicrobial agent.
